(19) United States
(12) Patent Application Publication
(10) Pub. No.: US 2016/0287337 A1
(43) Pub. Date: Oct. 6, 2016

(54) ORTHOPAEDIC SURGICAL SYSTEM AND METHOD FOR PATIENT-SPECIFIC SURGICAL PROCEDURE

(71) Applicants: Luke J. Aram, Warsaw, IN (US); Dan Fritzinger, Warsaw, IN (US); David Yingling, Warsaw, IN (US); Patrick Courtis, Winona Lake, IN (US); Daniel E. Lashure, Fort Wayne, IN (US)

(72) Inventors: Luke J. Aram, Warsaw, IN (US); Dan Fritzinger, Warsaw, IN (US); David Yingling, Warsaw, IN (US); Patrick Courtis, Winona Lake, IN (US); Daniel E. Lashure, Fort Wayne, IN (US)

(21) Appl. No.: 15/086,911

(22) Filed: Mar. 31, 2016

Related U.S. Application Data
(60) Provisional application No. 62/140,855, filed on Mar. 31, 2015.

(51) Int. Cl. A61B 34/10

(52) U.S. Cl. CPC A61B 34/10; A61B 2034/107; A61B 2034/108; A61B 2034/252

(57) ABSTRACT

A system and method for a patient-specific surgical procedure is disclosed. The method may include preparing a request for a customized surgical plan, loading the customized surgical plan onto an augmented reality device, and performing an orthopaedic surgical procedure with the augmented reality device in accordance with the customized surgical plan.

This application claims priority under 35 U.S.C. §119 to U.S. Patent App. Ser. No. 62/140,855, which was filed on Mar. 31, 2015 and is expressly incorporated herein by reference.

### TECHNICAL FIELD

The present disclosure relates generally to orthopaedic surgical instrument systems, and in particular to surgical instrument systems that utilize customized surgical plans in patient-specific surgical procedures.

### BACKGROUND

Joint arthroplasty is a well-known surgical procedure by which a diseased and/or damaged natural joint is replaced by a prosthetic joint. The prosthetic joint may include a prosthesis that is implanted into one or more of the patient's bones. For example, the orthopaedic prosthesis may replace the patient's knee, hip, shoulder, ankle, or other joint. In the case of a knee replacement, the orthopaedic knee prosthesis may include a tibial tray, a femoral component, and a polymer insert or bearing positioned between the tibial tray and the femoral component. In some cases, the knee prosthesis may also include a prosthetic patella component, which is secured to a posterior side of the patient's surgically-prepared patella.

In the case of a hip prosthesis, the orthopaedic hip prosthesis may include a femoral prosthesis that is implanted into a patient's femur. A femoral prosthesis typically includes an elongated stem component that is received in the medullary canal of the patient's femur and a spherically-shaped head component that bears against the patient's acetabulum or a prosthetic replacement acetabular cup.

The elongated stem implant may be cemented into the medullary canal or may have a porous coated surface for allowing the bone to heal directly to the implant. In some prostheses, the head is attached to a neck of the elongated stem via a taper connection. It is important to assemble the head to the neck with enough force so as to limit micromotion between the head and neck. The acetabulum of the patient may also be reamed to receive a shell and liner. A polyethylene, metal or ceramic liner with a metal shell is inserted into the acetabulum and acts as socket for receiving the head on the stemmed implant.

To facilitate the replacement of the natural joint with an orthopaedic prosthesis, orthopaedic surgeons use a variety of orthopaedic surgical instruments such as, for example, cutting blocks, drill guides, milling guides, and other surgical instruments. Such orthopaedic surgical instruments may be generic or specific to the patient.

Various techniques exist for utilizing a surgical plan that has been customized for a particular patient, including those described in U.S. Patent App. Pub. Nos. 2013/0261503 and 2009/0089081, which are expressly incorporated herein by reference.

During the performance of a typical orthopaedic surgical procedure, an orthopaedic surgeon may rely on a broad range of orthopaedic surgical information. Such orthopaedic surgical information may include pre-operative notes and diagrams, patient X-rays and historical data, navigational data, surgical procedure images, data obtained from various sensors, and other data related to the orthopaedic surgical procedure and/or patient. Various systems and techniques exist for providing information to a surgeon or other personnel during a surgical procedure, including those described in U.S. Pat. No. 5,526,812 and U.S. Patent App. Pub. Nos. 2007/0015999 and 2006/0142739, which are expressly incorporated herein by reference.

### SUMMARY

According to one aspect of the disclosure, a method for a patient-specific surgical procedure is disclosed. The method includes preparing a request for a customized surgical plan, receiving the customized surgical plan from a vendor, loading the customized surgical plan onto an augmented reality device, and performing an orthopaedic surgical procedure with the augmented reality device in accordance with the customized surgical plan. Performing the orthopaedic surgical procedure includes selecting a surgical instrument based on information displayed by the augmented reality device and operating the surgical instrument based on visual indications provided by the augmented reality device.

In some embodiments, the visual indications provided by the augmented reality device may include a digital template of the patient's bone overlaying the patient's actual bone.

In some embodiments, the augmented reality device may be operable to change the digital template when the surgical instrument is properly positioned relative to the patient's bone.

In some embodiments, the augmented reality device may be operable to change a color of the digital template when the surgical instrument is properly positioned relative to the patient's bone.

Additionally, performing the orthopaedic surgical procedure may include positioning the surgical instrument to permit the augmented reality device to identify the surgical instrument.

In some embodiments, performing the orthopaedic surgical procedure with the augmented reality device may include accessing the customized surgical plan on the augmented reality device to display a three dimensional rendered image of a patient's bone. Additionally, in some embodiments, accessing the customized surgical plan on the augmented reality device to display the three dimensional rendered image of the patient's bone may include displaying a digital template of the patient's bone overlaying the patient's actual bone on a display of the augmented reality device.

In some embodiments, the method may include operating the augmented reality device to change the display of the augmented reality device when the surgical instrument is properly positioned relative to the patient's bone. Additionally, in some embodiments, operating the augmented reality device to change the display may include changing a color of the digital template when the surgical instrument is properly positioned relative to the patient's bone.

According to another aspect, a method of utilizing a patient-specific surgical plan is disclosed. The method includes operating a display to provide surgical plan information, identifying a surgical instrument, updating the surgical plan information based on the surgical instrument, monitoring the position of the surgical instrument, and providing an indication to the user when the surgical instrument is properly positioned relative to the patient's bone. In some embodiments, the indication may be a visual indication.

In some embodiments, the visual indication may include a digital template of a patient's bone.

In some embodiments, the surgical plan information may include patient-specific medical images.

In some embodiments, providing the visual indication may include changing an outline of the digital template to indicate to the user when the surgical instrument is properly positioned relative to the patient's bone.

In some embodiments, the patient's bone may be a tibia. In some embodiments, the patient's bone may be a femur. In some embodiments, the patient's bone may be a pelvic bone.

According to another aspect, a system for performing any of the methods described herein is disclosed. The system for utilizing a patient-specific surgical plan may comprises a surgical instrument including a data tag, and an augmented reality device including a display, a sensor operable to scan the data tag and generate an electronic signal, and a controller operable to receive the electronic signal, determine the position of the surgical instrument based on the electronic signal, and operate the display to provide an indication to the user when the surgical instrument is properly positioned relative to a patient's bone.

In some embodiments, the controller may be operable to operate the display to overlie a digital template of the patient's bone on a patient's actual bone. In some embodiments, the controller may be operable to operate the display to change the digital template when the surgical instrument is properly positioned relative to the patient's bone. According to another aspect, an orthopaedic surgical instrument for use with the system is disclosed.

### BRIEF DESCRIPTION OF THE DRAWINGS

The detailed description particularly refers to the following figures, in which:

FIG. 1 illustrates a system for generating and utilizing a surgical plan that has been customized for a particular patient;

FIG. 2 is a perspective view of one embodiment of an augmented reality device for use during a surgical procedure;

FIG. 3 is a plan view of the augmented reality device of FIG. 2;

FIG. 4 is a perspective view of a surgical instrument for use during a surgical procedure;

FIGS. 5-6 are block diagrams of a method for generating and performing a custom surgical plan; and

FIGS. 7-22 are illustrations of various steps of a custom surgical plan utilizing the augmented reality device of FIG. 2.

### DETAILED DESCRIPTION OF THE DRAWINGS

While the concepts of the present disclosure are susceptible to various modifications and alternative forms, specific exemplary embodiments thereof have been shown by way of example in the drawings and will herein be described in detail. It should be understood, however, that there is no intent to limit the concepts of the present disclosure to the particular forms disclosed, but on the contrary, the intention is to cover all modifications, equivalents, and alternatives falling within the spirit and scope of the invention as defined by the appended claims.

Terms representing anatomical references, such as anterior, posterior, medial, lateral, superior, inferior, etcetera, may be used throughout the specification in reference to the orthopaedic implants or prostheses and surgical instruments described herein as well as in reference to the patient's natural anatomy. Such terms have well-understood meanings in both the study of anatomy and the field of orthopaedics. Use of such anatomical reference terms in the written description and claims is intended to be consistent with their well-understood meanings unless noted otherwise.

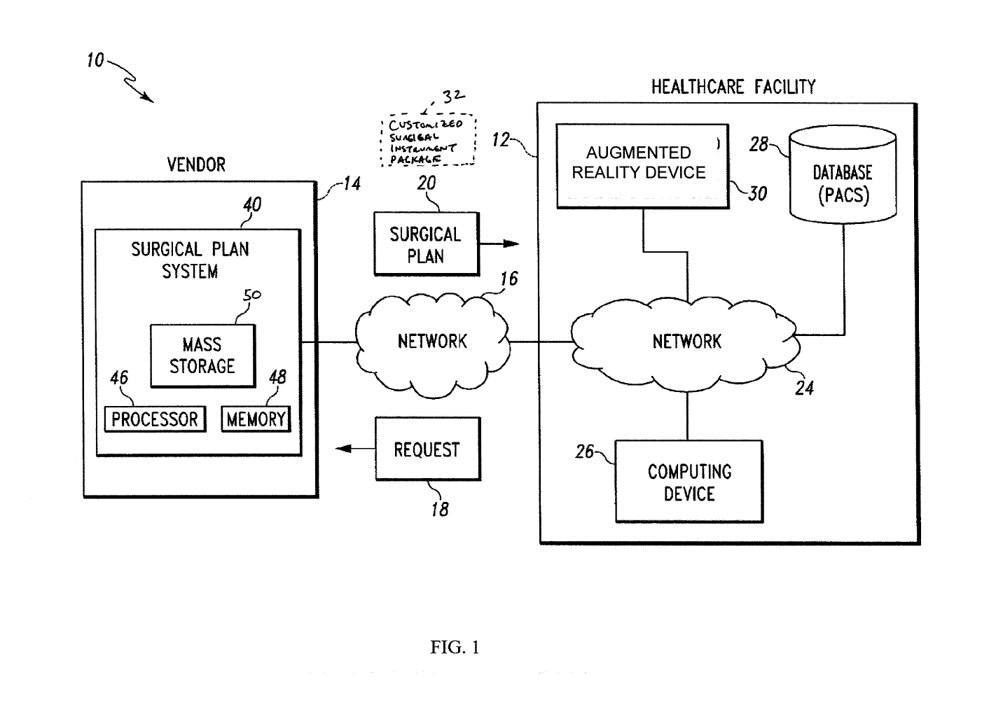

Referring to FIG. 1, an orthopaedic surgical system 10 for customizing and utilizing surgical plans in a surgical procedure is shown. In the illustrative embodiment, the system 10 includes a healthcare facility 12, a vendor 14 that provides a custom surgical plan 20, and a network 16 that connects the healthcare facility 12 to the vendor 14. As described in greater detail below, the healthcare facility 12 includes one or more computing devices 26 that may be used to prepare a request 18 for a surgical plan 20 based on patient information stored in a medical database 28. In response, the vendor 14 provides the surgical plan 20, which may uploaded into an augmented reality device 30 that may be used during the surgical procedure to provide information to surgeon during the performance of a surgical procedure. In some cases, the vendor 14 may also provide a customized package 32 of surgical instruments for use with the plan 20.

The networks 16, 24 may include one or more wide area networks (WAN), local area networks (LAN), and/or publicly-accessible global networks such as, for example, the Internet. In addition, the networks 16, 24 may include one or more wired networks and/or wireless networks. As such, the network 16 may include routers, switches, computers, communication links, and other networking components that cooperate to connect the vendor 14 and the healthcare facility 12. The network 24 may include similar devices that cooperate to connect the computing device 26, databases 28, the augmented reality devices 30, and other devices to facilitate communication between those devices.

The computing devices 26 are operable to display data and receive input from caregivers of the healthcare facility 12 such as, for example, doctors, nurses, anesthesiologists, and surgeons. The computing devices 26 may include a variety of different computing devices such as, for example, servers, desktop computers, laptop computers, handheld computers, personal data assistants, mobile phones, and possibly other computing devices. A computing device 26 is illustrated in FIG. 1 as being physically located within the healthcare facility 12; however, in some embodiments, one or more of the computing devices 26 may remotely access the healthcare facility network 24 from locations external to the healthcare facility 12. Such embodiments may enable caregivers to order and/or otherwise define custom surgical plans 20 while the caregiver is away from the healthcare facility 12.

The databases 28 may store personal data, medical data, and/or other data associated with patients of the healthcare facility 12. In one embodiment, the databases 28 may include a Patient Archiving Communications System (PACS) that stores medical images for patients of the healthcare facility 12. Those medical images may include X-ray images, magnetic resonance imaging (MRI) images, computerized tomography (CT) images, or the like. The databases 28 may also include data relevant to the particular medical images, including, for example, the patient's name and other patient identification information, date of the images, surgeon's or doctor's name, the name of the hospital or healthcare facility wherein the medical images were generated, and the like.

As shown in FIG. 1, the vendor 14 has a surgical plan system 40 that may receive a request 18 for a surgical plan via network 16 from the healthcare facility 12. The vendor 14 may utilize the system 40 to generate a surgical plan 20 that has been customized based upon information of the received request, and provide the healthcare facility 12 with the custom surgical plan 20 via network 16. The surgical plan system 40 may include one or more computing devices and associated software, middleware, and/or firmware that cooperate to customize the surgical plan as described herein, including, for example, one or more microprocessors 46, one or more memory devices 48, and one or more mass storage devices 50.

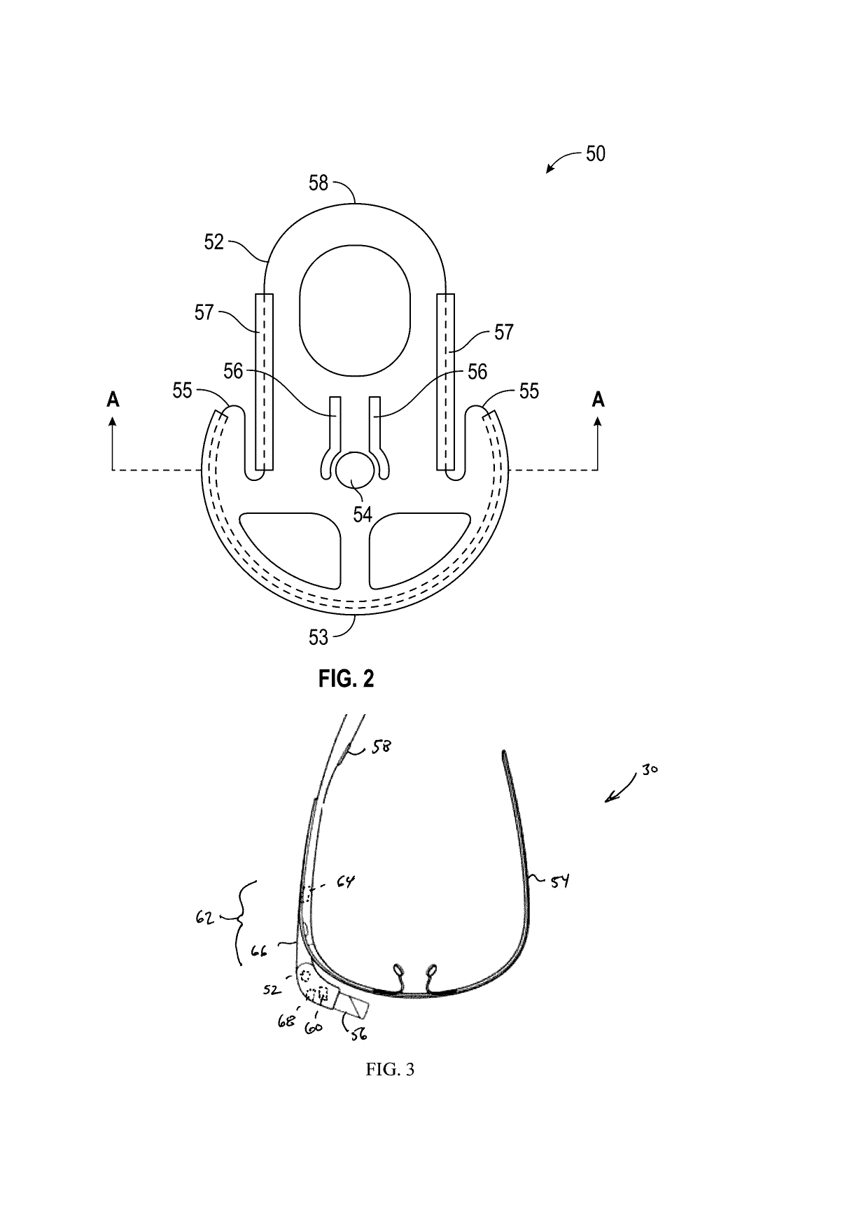

As described above, the system 10 also includes an augmented reality (“AR”) device 30 that may be utilized during a surgical procedure to provide information to surgeon during the performance of a surgical procedure. In the illustrative embodiment, the AR device 30 is operable to display the various steps of the customized surgical plan along with other data that may be useful to the surgeon during the performance of the surgical procedure. As shown in FIG. 2, the AR device 30 is configured to be worn by a user. One example of an AR device 30 is the Google Glass, but it should be appreciated that the AR device 30 may take the form of other optical head-mounted displays and other types of wearable computers.

As shown in FIG. 3, the AR device 30 includes a number of electronic components found in a typical computer system, including a microprocessor 52 or other central processing unit and memory, which are mounted in a frame 54. The AR device 30 also includes a display 56 that is positioned at eye level when the AR device 30 is worn by the user. In the illustrative embodiment the display 56 is a Liquid Crystal on Silicon display operable to display images to the surgeon. A speaker 58 is secured to the frame 54 and is configured to be positioned adjacent to an ear of the user to provide audible information.

The AR device 30 includes a wireless transceiver 60 operable to be connected to the network 24. The transceiver 60 is illustratively designed to communicate via Wi-Fi, but it should be appreciated that other wireless communication protocols such, as for example, Wireless USB, Bluetooth, and the like may be used. It should also be appreciated that in other embodiments the AR device 30 may utilize a wired communication protocol.

The AR device 30 includes a number of user-operated controls 62 that permit the user to control the electronic components of the AR device 30. The user-operated controls 62 include a microphone 64 and a touchpad 66, but it should be appreciated that in other embodiments the controls 62 may include any combination of buttons, motion sensors, or other controls. The AR device 30 also includes a sensor 68, which is illustratively embodied as a camera 68 operable to, among other things, take photos, record video information, and provide video data to the other electronic components of the AR device 30.

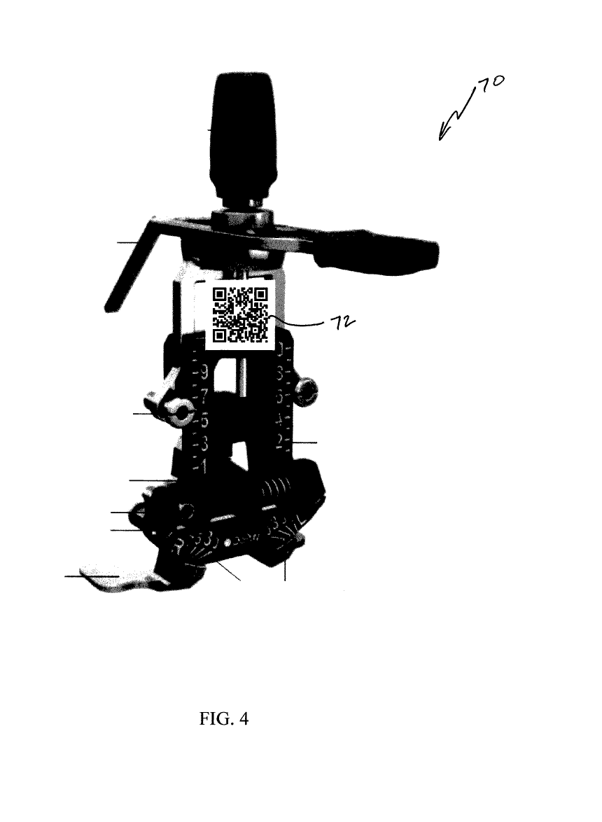

Referring now to FIG. 4, the system 10 includes a number of surgical instruments 70 for use during the surgical procedure to prepare the patient's bone to receive an orthopaedic prosthesis. The instruments 70 may be included in a customized package 32 of the surgical instruments for use with a particular patient or may be included in a generic instrument package. In the illustrative embodiment, a data tag 72 may be attached to the surgical instrument 70, as shown in FIG. 4. The data tag 72 contains information, which, among other things, permits the surgical instrument 70 to be identified by the AR device 30. The tag 72 is attached to, embedded in, or otherwise secured to the surgical instrument 70. In other embodiments, the data tag 72 may be incorporated into the packaging of the surgical instrument 70.

The data tag 72 may be embodied as any type of data tag capable of being read by the AR device 30 to provide information about the item to which it is attached. For example, as shown in FIG. 4, the data tag 72 includes a Quick Response Code (QR Code). However, in other embodiments, other types of data tags may be used such as, for example, barcodes, matrix barcodes, or other type of electrical or printed data tag. In still other embodiments, the data tag may be a radio frequency identification (RFID) tag.

In use, a surgeon may use the computing device 26 to send a request 18 to a vendor 14 to generate a customized surgical plan 20, which is sent to the healthcare facility 12. The vendor 14 may also prepare a customized instrument package 32 for use with the surgical plan 20. Such a customized instrument package 32 may include one or more customized patient-specific surgical instruments. As used herein, the term “customized patient-specific orthopaedic surgical instrument” is distinct from orthopaedic prostheses, whether patient-specific or generic, which are surgically implanted in the body of the patient. Rather, customized patient-specific orthopaedic surgical instruments are used by an orthopaedic surgeon to assist in the implantation of orthopaedic prostheses. Exemplary customized patient-specific surgical instruments are shown and described in U.S. Pat. No. 8,425,523, which is incorporated herein by reference. The customized instrument package 32 may also include a reduced set of generic surgical instruments that are selected based on the particular patient. For example, the vendor 14 may include a more limited range of prosthetic trial sizes based on the size of the patient.

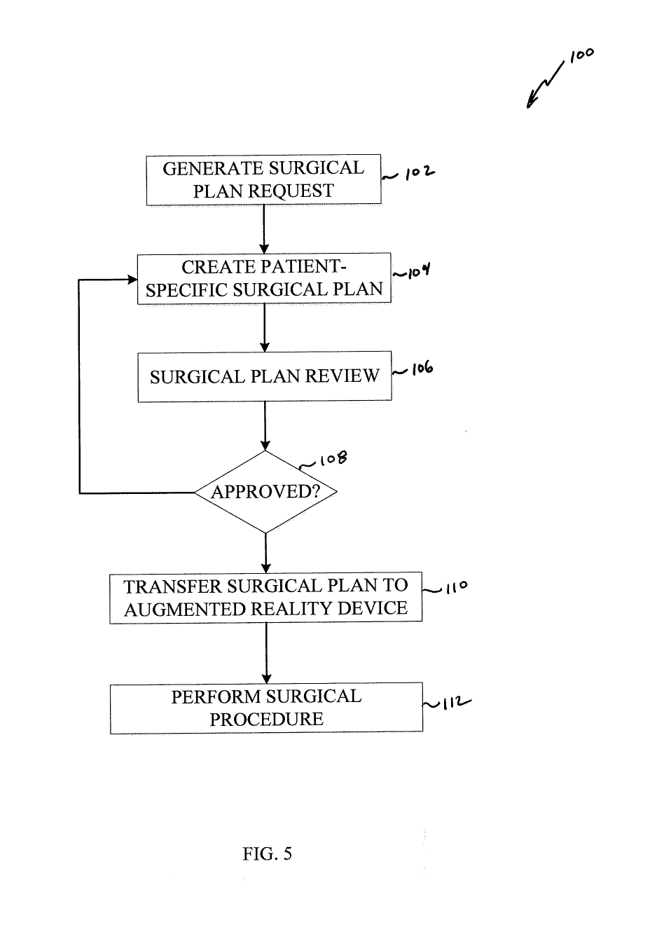

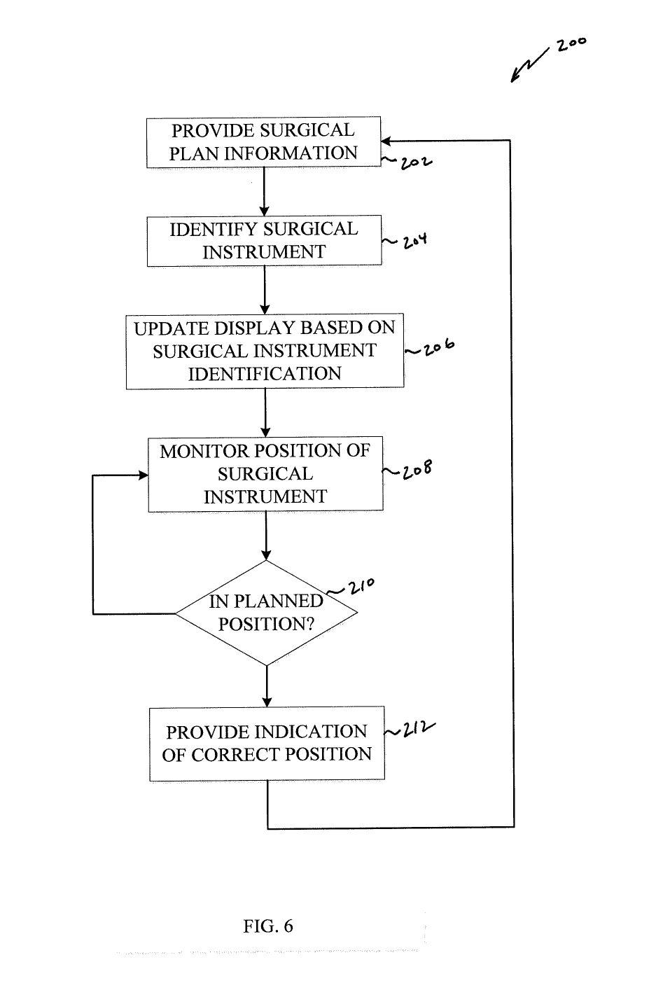

Referring now to FIGS. 5-6, a method 100 for generating and utilizing a customized surgical plan 20 in a surgical procedure is shown. The method 100 may begin in block 102 with the surgeon or other user defining a surgical plan request 18 for a particular patient. To that end, the surgeon may use the computing device 26 to enter or otherwise collect data that is relevant to the planned surgical procedure and the particular patient. The data may include, but is not limited to, the type of orthopaedic surgical procedure to be performed, the type of orthopaedic implant to be used, rendered images of the relevant anatomical portions of the patient, digital templates of the orthopaedic implants and/or planned resection lines, pre-operative notes, diagrams, historic patient data, X-rays and other medical images, patient medical records, patient identification data, and/or any other data useful for customizing the orthopaedic surgical procedure to the patient.

Typically, medical images are generated pre-operatively in preparation for an orthopaedic surgical procedure. The medical images may include a medical image of the relevant bone(s) taken along the sagittal plane of the patient's body and a medical image of the relevant bone(s) taken along the coronal plane of the patient's body. The medical images may include X-ray images, magnetic resonance imaging (MRI) images, computerized tomography (CT) images, and/or any other type of image capable of providing indicia of the relevant bone or bones. Such imaging devices may be located in the healthcare facility 12 or may be located remote therefrom. The imaging devices may or may not be communicatively coupled to the healthcare facility 12.

In addition, the surgeon may enter constraint data that may limit, restrict, or otherwise affect the generation of the surgical plan 20 for the patient. For example, the constraint data may include the surgeon's preference for an orthopaedic implant type, the surgeon's preference for particular parts of the implant, the degree of acceptable orthopedic implant sizes (e.g., a restriction on the range of implant sizes that may be recommended), the amount of bone that will be resected, the planned location and/or orientation of the orthopaedic implant, fixation type (e.g., cement or cementless), material type, finish, and other features such as head size and other preferences such as metal-on-metal, metal-on-ceramic, ceramic-on-ceramic, metal-on-poly, or the like. The constraint data may also identify a branded surgical procedure that has been branded or otherwise associated with a particular surgeon, healthcare facility, university and/or another person or institution. For example, a surgeon may request a surgical plan 20 for a knee replacement in the style of a prominent doctor or institution.

When the surgeon has completed the plan request 18, it is transmitted to the vendor 14. In block 104, the vendor 14 utilizes the surgical plan system 40 to create a surgical plan 20 based upon the constraints and other data provided by the surgical plan request 18. In creating the surgical plan 20, the surgical plan system 40 may perform a digital templating procedure on medical images of the surgical plan request 18 to determine an orthopaedic implant to recommend to the surgeon or healthcare facility for use with relevant bone(s) of the patient. To do so, the surgical plan system 40 may be configured to determine relevant aspects of the patient's bone or bony anatomy from the medical images. For example, the surgical plan system 40 may determine one or more mechanical axis of the relevant bones, determine one or more resection planes of the relevant bones, locate particular features of the relevant bones, and/or the like. Based on such determinations, the surgical plan system 40 may select an appropriate orthopaedic implant type and size that also satisfies the implant constraint data of the surgical plan request 18. The surgical plan system 40 may also be configured to determine recommended range of sizes of the orthopaedic implant. For example, in some embodiments, the surgical plan system 40 may be configured to determine an orthopaedic implant within a range of plus or minus two sizes. For example, the surgical plan system 40 may recommend an orthopaedic implant of a size 5+/−2 sizes (i.e., a range of size 3 to size 7).

In creating the surgical plan 20, the surgical plan system 40 may also retrieve a digital template(s) of the orthopaedic implant and the orthopaedic surgical instruments that may be used to prepare the patient's bone. The digital template may be retrieved from, for example, the memory device 48, mass storage device 50 or from any other storage location capable of storing a number of digital templates. The digital template may include one or more two-dimensional and/or three-dimensional electronic renderings of the orthopaedic implant or surgical instrument(s) selected for the surgical procedure, or components thereof, that is capable of being superimposed on a medical image of the patient. For example, a digital template may be embodied as a two-dimensional or three-dimensional electronic rendering of a surgical instrument that is capable of being superimposed or otherwise incorporated into a medical image of a tibia or femur bone of the patient. As discussed in more detail below, the digital template may be used in conjunction with indicia of the determined aspects or features of the relevant bones such as lines or other indicia of the mechanical axis or resection points/planes of the relevant bones.

The surgical plan system 40 may then superimpose or otherwise incorporate the digital template into the medical images of the surgical plan request 20. The digital template may be a two-dimensional electronic rendering and/or three-dimensional electronic rendering of the orthopaedic implant or surgical instrument. To do so, the surgical plan system 40 may be configured to use any suitable algorithm and data of the surgical plan request 18 to determine a recommended location and orientation of the orthopaedic implant, as represented by the digital template, with respect to the patient's bone. For example, if implant constraint data provides an estimated amount of resection of the patients' bone, the surgical plan system 40 may be configured to position the digital template of the orthopaedic implant in the medical images based on such estimated resection. Similarly, the surgical plan system 40 may be configured to position the digital template of a surgical instrument, such as, for example, a resection guide, in the medical images in order to create the estimated resection.

In addition, any one or more of the aspects of the bone as determined above may be used to determine the proper positioning of the digital template. For example, the determined mechanical axis, resection planes, and/or other determined aspects of the relevant bones may be used to determined the proper positioning of the digital templates. In this way, the surgical plan system generates a number of digital templated medical images having indicia of the relevant bone's of the patient and indicia of the recommended orthopaedic implant and/or surgical instruments positioned in a location and orientation.

Although described above as an automated process, in some embodiments, the creation of the surgical plan 20 may be a semi-automated or a manual process. For example, a technician such as a CAD operator or medical technician of the vendor 14 may determine the aspects of the relevant bones such as the mechanical axis, the resection lines, and/or other particular features of the relevant bones. The technician may edit the medical images such that indicia of such aspects are superimposed or otherwise incorporated into the medical images. For example, the technician may electronically draw the mechanical axes and/or resection lines. Once such aspects of the relevant bones are determined, the surgical plan system 40 may be configured to determine a recommended orthopaedic implant or surgical instrument and create a custom surgical plan 20 based on the aspects of the relevant bones determined by the technician, retrieve a digital template of the recommended orthopaedic implant and/or surgical instrument, and superimpose or otherwise incorporate the digital template into the medical image based on, for example, the determined aspects of the relevant bones.

In other embodiments, the custom surgical plan 20 may be manually created by the vendor 14. In such embodiments, a technician may determine the aspects of the relevant bones, incorporate indicia of such aspects into the medical images (e.g., draw the mechanical axis, resection lines, etc. of the relevant bones), and determine a recommended orthopaedic implant and surgical plan 20 based on such aspects. The technician may then manually superimpose or otherwise incorporate a digital template of the recommended orthopaedic implant or surgical instrument into the medical images. The technician may locate and orientate the digital template based on the determined aspects of the relevant bones. For example, the technician may position the digital template of the recommended orthopaedic implant or surgical instrument in the medical image using a suitable CAD software program or the like.

Regardless, the surgical plan system 40 may then transfer the surgical plan 20 to the healthcare facility 12. At block 106, the surgeon may review the surgical plan via the computer device 26. For example, the surgeon may review the digital templated medical images to determine if the digital templates of the orthopaedic implant and surgical instruments are properly located with respect to the patient's bone, if the type of recommended orthopaedic implant and/or surgical instrument is correct, if a larger or smaller orthopaedic implant size is more desirable, and/or the like.

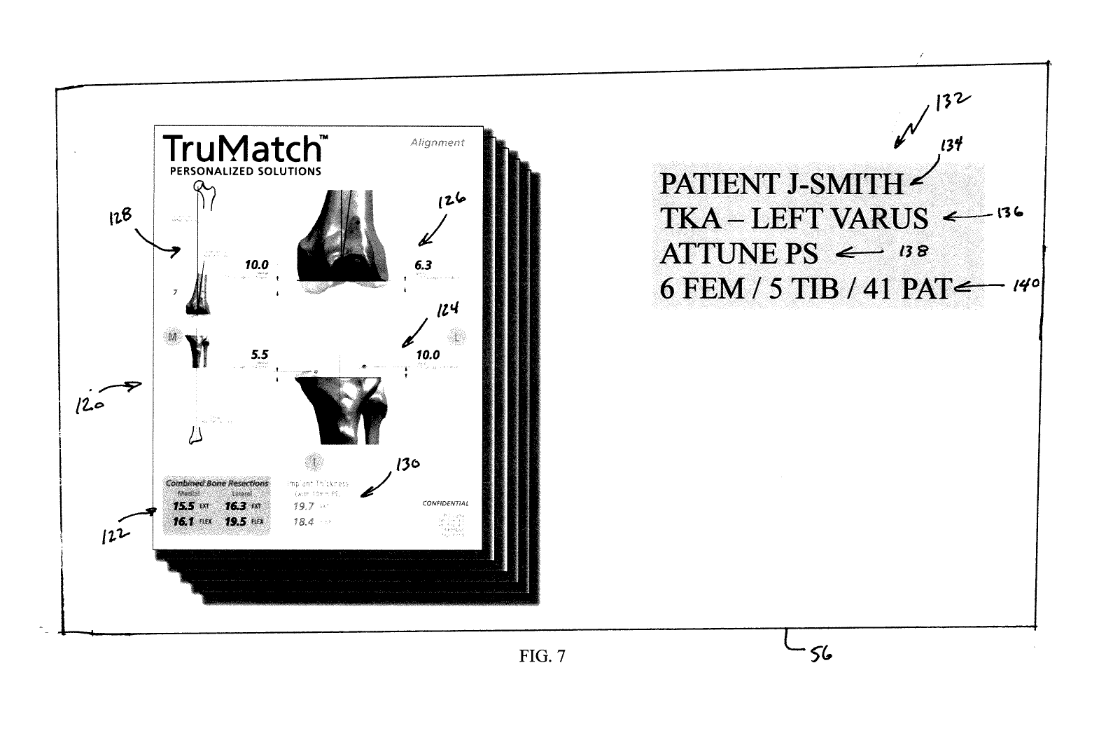

At the same time, the surgeon may review the series of procedural steps defined by the surgical plan 20. The procedural steps may be included in a technique or guide such as the guide 120 shown in FIG. 7. In the illustrative embodiment, the guide 120 includes surgical procedure parameters related to a total knee arthroplasty surgery and identifies, for example, combined bone resections 122, the level of resection 124 for the tibia, and the level of resection 126 for the femur. The guide 120 also includes information related to the planned alignment 128 of the tibia and femur as well as the planned implant thickness 130. In other embodiments, the guide may include any additional or other data related to any aspect of the specific orthopaedic surgical procedure, the surgical plan, the orthopaedic implant, the orthopaedic surgical instrument, the patient, the orthopaedic surgeon or healthcare facility, and/or any other data relevant to and useful in the performance of the orthopaedic surgical procedure.

At block 108, the surgeon may decide based upon his review of the surgical plan 20 to have the vendor 14 modify the surgical plan 20. If the surgeon decides to have the vendor 14 change the surgical plan 20, the surgeon may modify aspects of the surgical plan request 18 such as, for example, providing further constraints or directives regarding the custom surgical plan 20 and transfer the modified surgical plan request 18 to the vendor 14. For example, the surgeon may modify a digital templated medical images and/or other data of the received surgical plan 20 and transmit the modified digital templated medical images and/or other data to the surgical plan system 40. The vendor 14 and the surgeon may then engage in an iterative process to refine the plan 20 until the surgeon approves of the plan.

If the surgeon is satisfied with the custom surgical plan 20 for the patient, the surgical plan 20 may be uploaded into the AR device 30 in block 110. To do so, the computing device 26 may receive the surgical plan 20 directly from the vendor 14. The surgeon or other user may then transfer the surgical plan 20 from the computing device 26 and/or database 28 of the healthcare facility 12 to the AR device 30 via the network 16. As described above, the vendor 14 may also prepare and ship a customized package 32 of the surgical instruments to the healthcare facility 12 for use with the surgical plan 20.

In block 112, the surgeon may operate the AR device 30 via controls 62 to retrieve the surgical plan 20 and other relevant data during the surgical procedure. The surgeon may access the data stored locally on the AR device 30 and, via the network 24, data stored on the databases 28 and/or computing devices 26. Such data might include pre-operative notes, diagrams, and other surgical plans, and images such as three dimensional rendered images of the relevant anatomical portions of the patient. The surgeon may also communicate with the vendor 14 or other surgeons via the network 16.

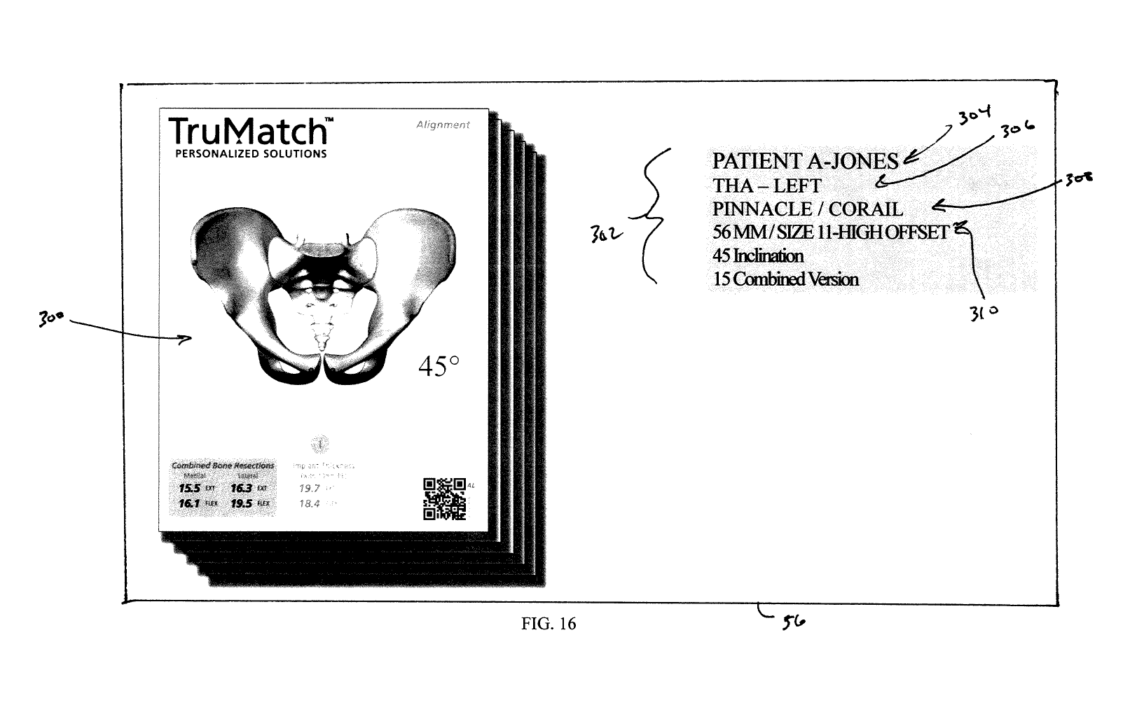

Referring now to FIG. 6, a routine 200 for utilizing the surgical plan 20 during an orthopaedic surgical procedure shown. The routine 200 may be a software program stored on the AR device 30, which may be activated the surgeon during the procedure to access the surgical plan 20. The routine 200 may provide surgical plan information to the surgeon in block 202. To do so, the processor 52 of the AR device 30 may activate the display 56 of the AR device 30. FIGS. 7-15 include illustrations of exemplary images displayed during a total knee arthroplasty procedure, while FIGS. 16-22 include illustrations of exemplary images displayed during a hip arthroplasty procedure, as described in greater detail below.

As shown in FIG. 7, the processor 52 in block 202 may operate the display 56 to present the surgeon with a page of the guide 120 and surgical procedure information 132. As described above, the guide 120 may include surgical procedure parameters related to a total knee arthroplasty surgery and may identify, for example, the combined bone resections 122, the level of resection 124 for the tibia, the level of resection 126 for the femur, and so forth. The surgical procedure information 132 may include patient information 134 and information 136, 138, 140 related to the type of procedure, the orthopaedic instrument system, and the proposed implant sizes, respectively.

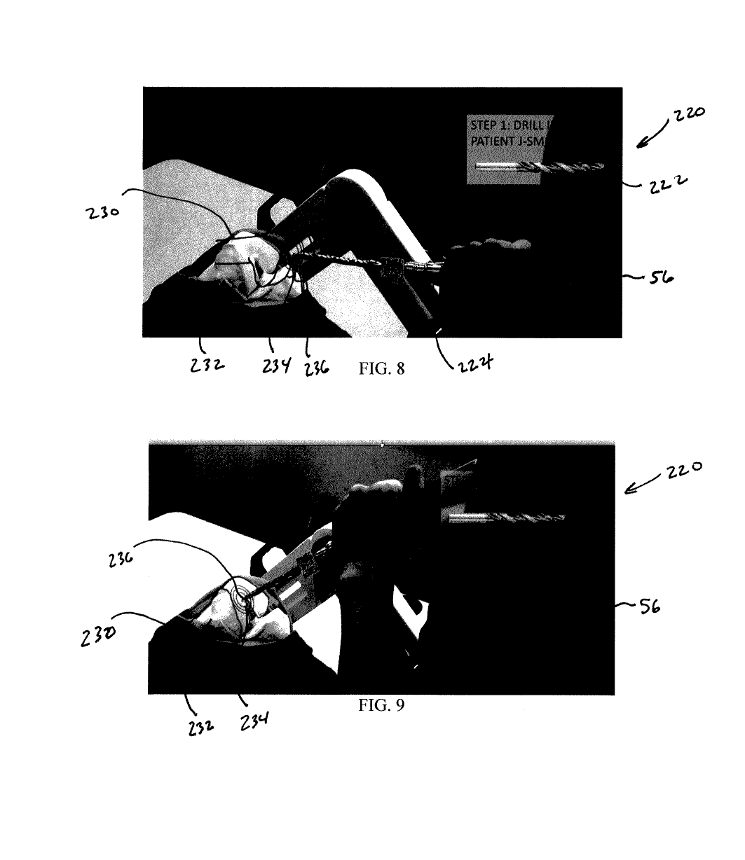

The surgeon may use one of the controls 62 to advance the routine 200 to the next block 204. In the block 204, the processor 52 accesses the surgical plan 20 to determine the next step of the surgical procedure. To do so, the processor 52 may access a procedure log stored in memory to determine what steps, if any, have already been performed. Based on that information, the processor 52 may activate the display 56 to provide the surgeon with information 220 about the next step of the surgical procedure, as shown in FIG. 8. That information may include an explanation of the next step as well as an image 222 of the surgical instrument to be used.

When the surgeon has selected a surgical instrument such as, for example, the surgical drill 224, the AR device 30 may verify the surgeon has selected the correct surgical instrument in block 204. To do so, the AR device 30 may scan the data tag 72 of the surgical drill 224 using image and/or video data from the camera 68. After the AR device 30 has confirmed that the surgeon has selected the correct instrument, the routine 200 may advance to block 206.

In block 206, the display 56 may be operated to display a digital template 230 of the patient's bone 232, as shown in FIG. 8. In the illustrative embodiment, the digital template 230 includes an outline of the bone 232 that visually indicates to the surgeon whether the surgical instrument 224 has been properly oriented relative to the bone 232. In the illustrative embodiment, the digital template 230 also includes a target zone 234 that identifies where the tip 236 of the drill 224 should be located prior to activating the drill.

In block 208, the AR device 30 monitors the position and orientation of the drill 224. To do so, the processor 52 accesses image and/or video data from the camera 68 and performs an image analysis to determine position and orientation of the drill 224. In some embodiments, the AR device 30 may also provide a visual indication via the display 56 or an audible indication via the speaker 58 to inform the surgeon when she is moving closer the proper orientation or further away.

In block 210, if the AR device 30 determines the drill 224 is not properly oriented, the routine 200 returns to block 208. If the AR device 30 determines the drill 224 is properly oriented, the routine 200 advances to block 212. In block 212, the processor 52 updates the display 56 to provide a visual indication that the drill 224 is properly oriented. For example, as shown in FIG. 9, the color of the digital template 230 changes from blue to green. In other embodiments, other aspects of the digital template 230 may change to provide the visual indication. In some embodiments, the AR device 30 may also provide or an audible indication via the speaker 58.

With the drill 224 properly oriented, the surgeon may proceed to define an opening in the patient's bone 232. In some embodiments, the processor 52 may be configured to deactivate the display 56 when the drill 224 is energized. In other embodiments, the processor 52 may provide a visual indication of when the drill 224 has reached the pre-planned depth in the patient's bone.

Returning to FIG. 6, the routine 200 may then return to block 202 following the completion of the drilling operation. To do so, the surgeon may operate one of the controls 62 to access the next step in the surgical plan 20. Alternatively, the AR device 30 may utilize the data from the camera 68 to determine that the surgeon has completed the current step of the procedure and automatically proceed to the next step of the procedure.

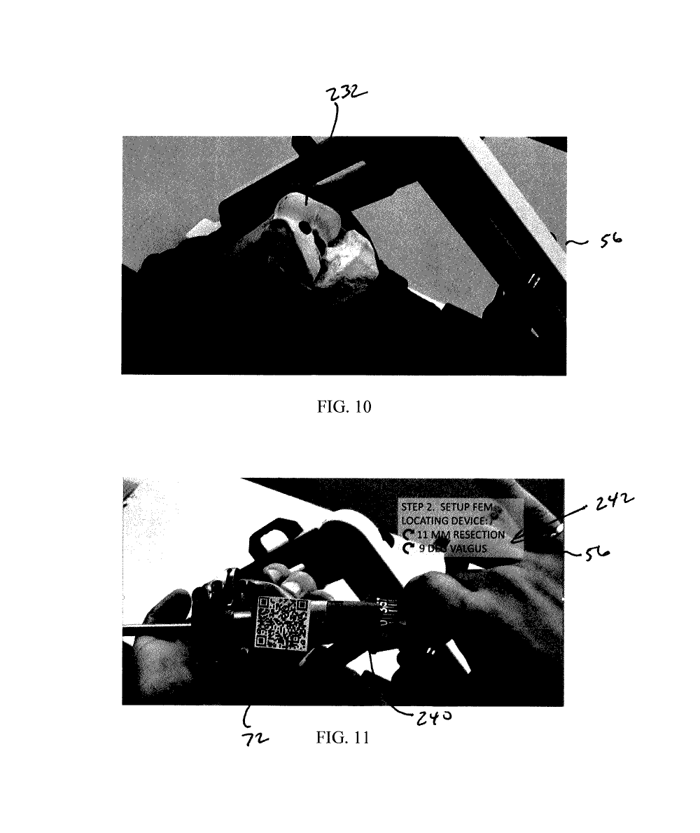

As shown in FIG. 10, the AR device 30 may be configured to pause after the surgeon has completed the drilling operation. When paused, the display 56 may be deactivated so that the surgeon may have an unobstructed view of the bone 232. As shown in FIG. 11, the surgeon may select the next surgical instrument 240. The AR device 30 may scan the data tag 72 of the surgical instrument 240 using image and/or video data from the camera 68 to identify the surgical instrument 240. After the AR device 30 has identified the surgical instrument 240, the processor 52 may activate the display 56 to provide information to the surgeon about the surgical instrument 240, including, for example, specific settings 242 of the surgical instrument 240, as shown in FIG. 11. The surgeon may then proceed to operate the adjustment knobs and other controls of the surgical instrument 240 to match the settings 242.

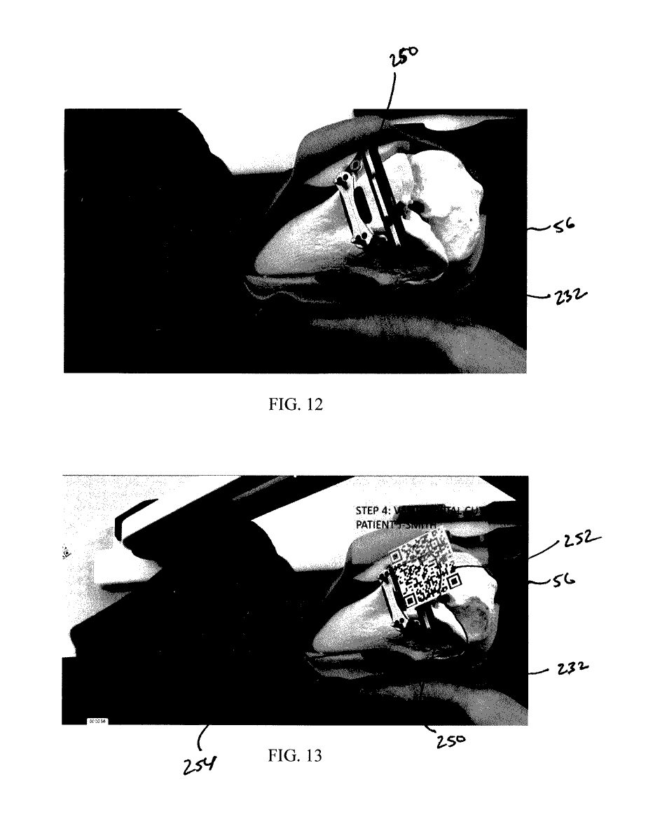

As shown in FIGS. 11-12, the AR device 30 may also be used to verify that a surgical instrument such as, for example, a cutting block 250, is properly positioned on the patient's bone. To do so, the surgeon may attach a data tag 252 to the cutting block 250, as shown in FIG. 12. The AR device 30 may scan the data tag 252 of the cutting block 250. After the AR device 30 has identified the cutting block 250, the processor 52 may operate the display 56 to overlay another digital template 254 of the patient's bone 232. When the digital template 254 is aligned with the bone 232 as shown in FIG. 13, the AR device 30 may provide a visual or audible indication to inform that surgeon that the cutting block 250 is properly positioned.

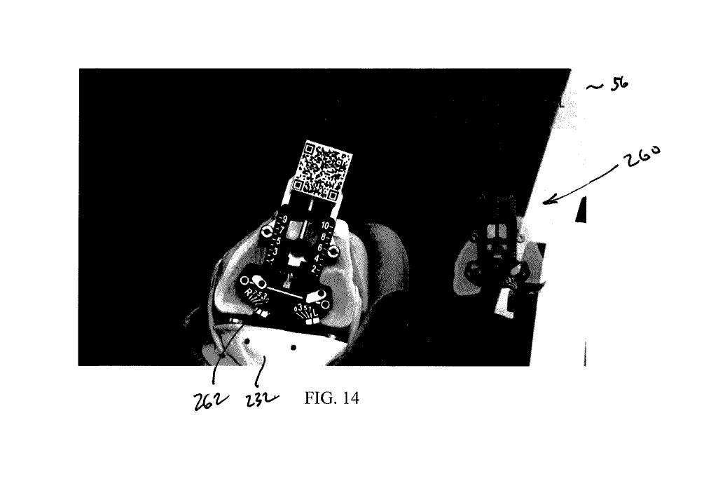

The AR device 30 may be configured to assist the surgeon in verifying the position of the surgical instruments in other ways. As shown in FIG. 14, for example, the AR device 30 may provide an alignment image 260 showing the surgical instrument 262 properly positioned on the bone 232. The surgeon may use the image 260 to perform a real-time comparison between the planned location and the instrument's actual location.

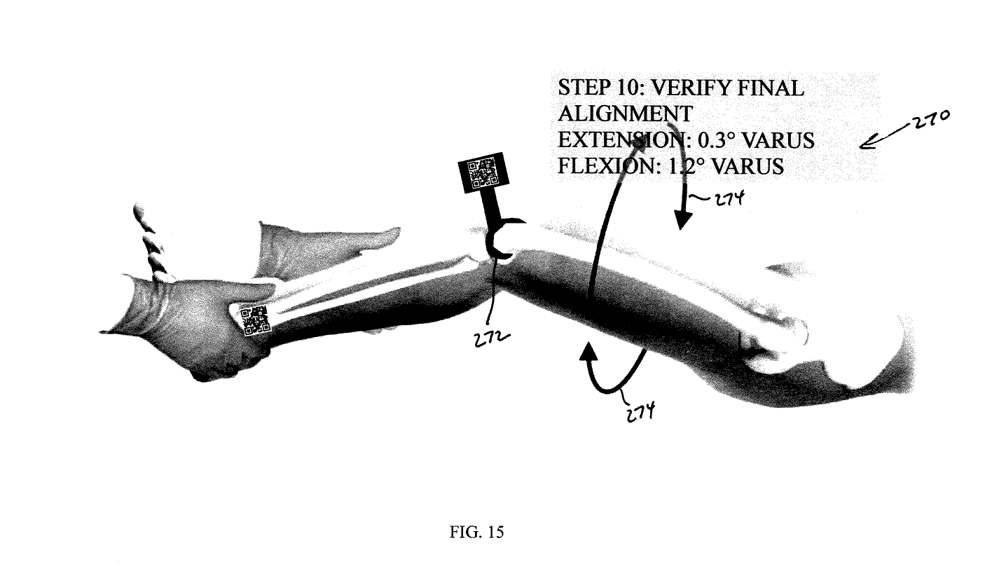

As shown in FIG. 15, the AR device 30 may also be configured to provide feedback to the surgeon during a trialing procedure. As shown in FIG. 15, the processor 52 operates the display 56 to provide target values 270 for extension and flexion as the surgeon confirms the final alignment. The AR device 30 also monitors the position and orientation of a femoral trial component 272. To do so, the processor 52 accesses image and/or video data from the camera 68 and performs an image analysis to determine position and orientation of the trial component 272. As shown in FIG. 15, the AR device 30 also provides a visual indication, i.e., arrows 274, via the display 56 to inform the surgeon when she is moving closer the proper orientation or further away.

As described above, FIGS. 16-22 include illustrations of exemplary images that the AR device 30 might display during a hip arthroplasty procedure when executing the routine 200. The routine 200 begins with block 202 in which the processor 52 may present a page of the guide 300 and surgical procedure information 302. The guide 300, like the guide 120, may include surgical procedure parameters relevant to the procedure. The surgical procedure information 302 may include patient information 304 and information 306, 308, 310 related to the type of procedure, the orthopaedic instrument system, and the proposed implant sizes, respectively.

The surgeon may use one of the controls 62 to advance the routine 200 to the next block 204. In the block 204, the processor 52 accesses the surgical plan 20 to determine the next step of the surgical procedure. To do so, the processor 52 may access a procedure log stored in memory to determine what steps, if any, have already been performed. Based on that information, the processor 52 may activate the display 56 to provide the surgeon with information 320 about the next step of the surgical procedure, as shown in FIG. 17. That information may include an explanation of the next step as well as an image 322 of the surgical instrument to be used.

When the surgeon has selected a surgical instrument such as, for example, the surgical broach 324, the AR device 30 may verify the surgeon has selected the correct surgical instrument in block 204. To do so, the AR device 30 may scan the data tag 72 of the broach 324 using image and/or video data from the camera 68. After the AR device 30 has confirmed that the surgeon has selected the correct instrument, the routine 200 may advance to block 206.

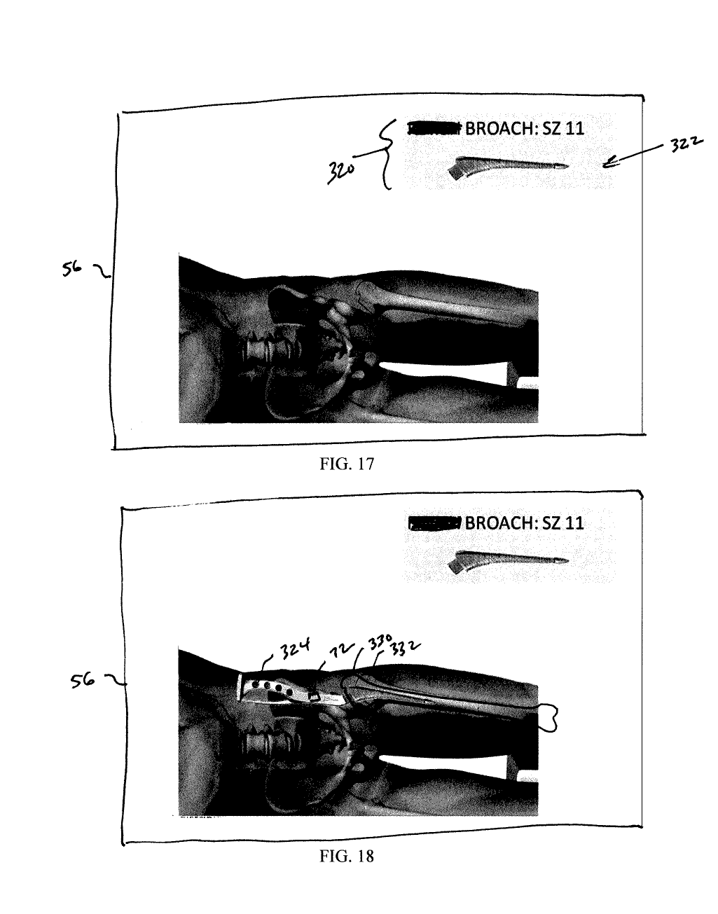

In block 206, the display 56 may be operated to display a digital template 330 of the patient's bone 332, as shown in FIG. 18. In the illustrative embodiment, the digital template 330 includes an outline of the bone 332 that visually indicates to the surgeon whether the broach 324 has been properly oriented relative to the bone 332.

In block 208, the AR device 30 monitors the position and orientation of the broach 324 as it is being advanced into the bone 332. To do so, the processor 52 accesses image and/or video data from the camera 68 and performs an image analysis to determine position and orientation of the broach 324. In some embodiments, the AR device 30 may also provide a visual indication via the display 56 or an audible indication via the speaker 58 to inform the surgeon when she is moving closer the pre-planned depth.

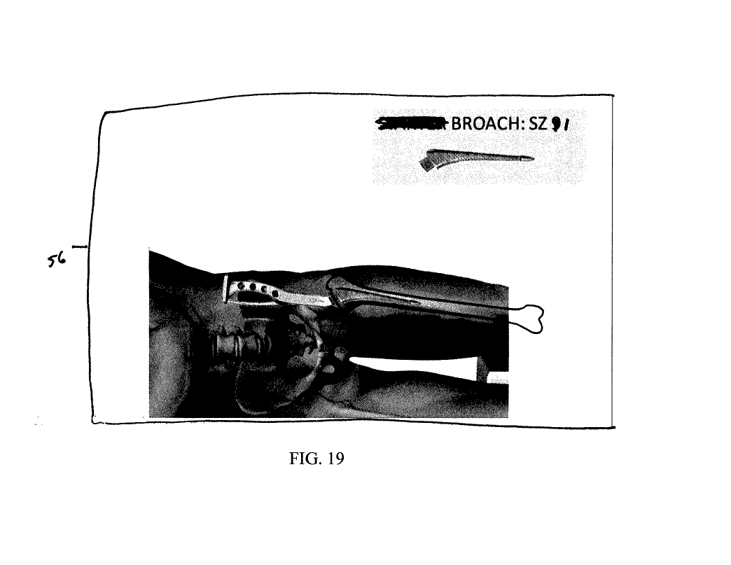

In block 210, if the AR device 30 determines the broach 324 has reached the pre-planned depth, the routine 200 returns to block 208. If the AR device 30 determines the broach 324 has reached the pre-planned depth, the routine 200 advances to block 212. In block 212, the processor 52 updates the display 56 to provide a visual indication that the broach 324 is properly oriented. For example, as shown in FIG. 19, the color of the digital template 330 changes from blue to green. In other embodiments, other aspects of the digital template 330 may change to provide the visual indication. In some embodiments, the AR device 30 may also provide or an audible indication via the speaker 58.

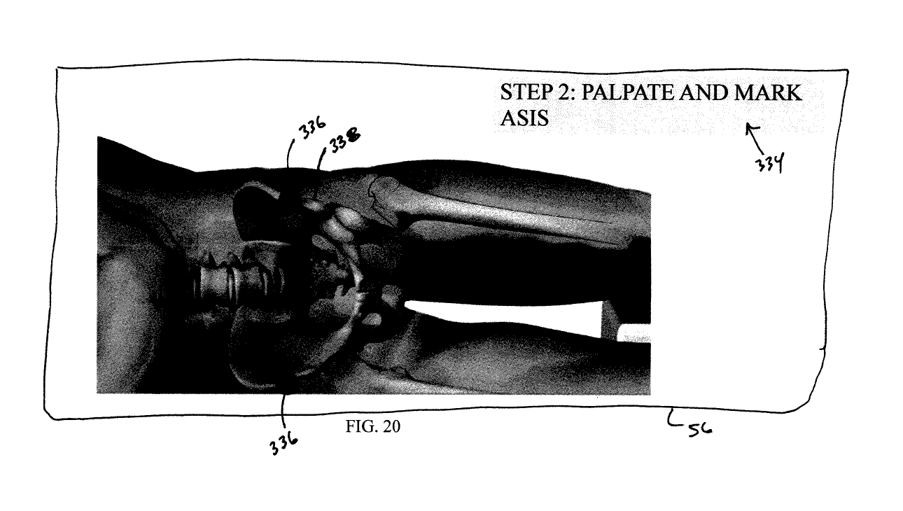

As described above, the routine 200 may then return to block 202 at the completion of block 210. Referring now to FIG. 20, the AR device 30 may proceed to the next step of the procedure. Again, the processor 52 may operate the display 56 to provide visual information 334 regarding the procedure step. The display 56 may also include digital markers 336 that overlay the patient's bone 338 to show where the surgeon should, for example, make a physical mark or place a physical marker 340 on the patient's bone.

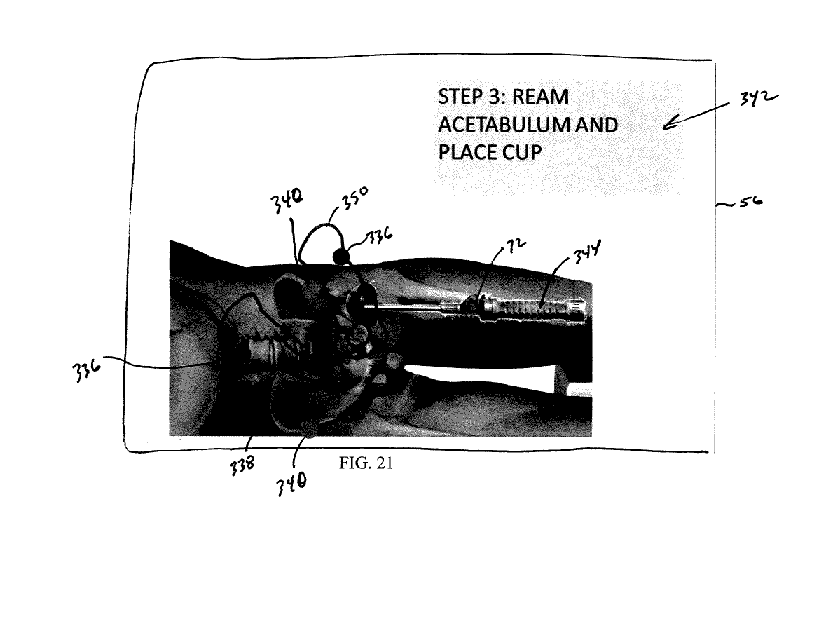

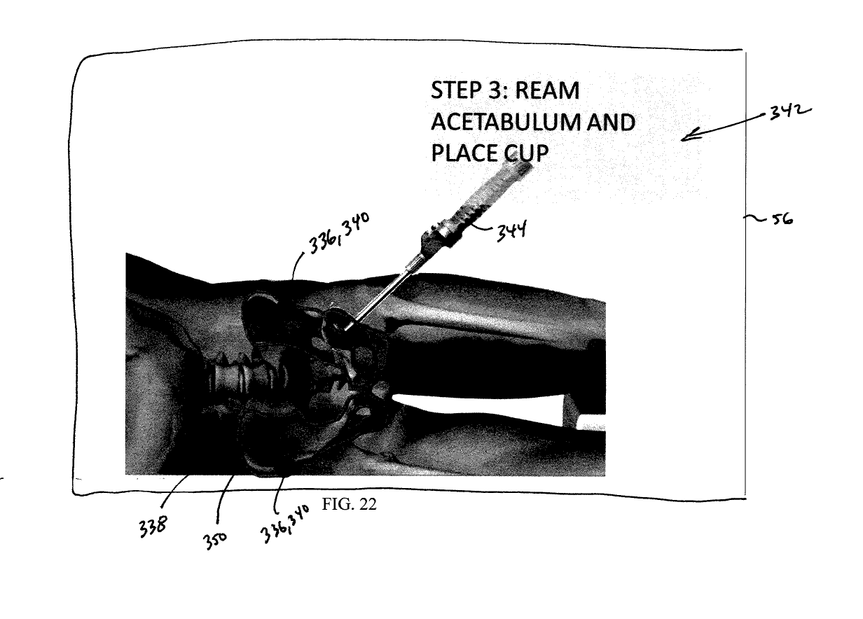

Referring now to FIGS. 21-22, the AR device 30 may proceed to the next step of the procedure and return to block 202 of the surgical procedure. The processor 52 may again activate the display 56 to provide the surgeon with information 342 about the next step of the surgical procedure, as shown in FIG. 21. When the surgeon has selected a surgical instrument such as, for example, the reamer 344, the AR device 30 may verify the surgeon has selected the correct surgical instrument in block 204. To do so, the AR device 30 may scan the data tag 72 of the reamer 344 using image and/or video data from the camera 68. After the AR device 30 has confirmed that the surgeon has selected the correct instrument, the routine 200 may advance to block 206.

In block 206, the display 56 may be operated to display a digital template 350 of the patient's bone 338, as shown in FIG. 21. In the illustrative embodiment, the digital template 350 includes an outline of the bone 338 that visually indicates to the surgeon whether the surgical instrument 344 has been properly oriented relative to the bone 338. In the illustrative embodiment, the digital template 350 also includes the markers 336 that correspond to the physical markers 340 on the patient's bone 338.

In block 208, the AR device 30 monitors the position and orientation of the reamer 344. To do so, the processor 52 accesses image and/or video data from the camera 68 and performs an image analysis to determine position and orientation of the reamer 344. In some embodiments, the AR device 30 may also provide a visual indication via the display 56 or an audible indication via the speaker 58 to inform the surgeon when she is moving closer the proper orientation or further away.

In block 210, if the AR device 30 determines the reamer 344 is not properly oriented, the routine 200 returns to block 208. If the AR device 30 determines the reamer 344 is properly oriented, the routine 200 advances to block 212. In block 212, the processor 52 updates the display 56 to provide a visual indication that the reamer 344 is properly oriented. For example, as shown in FIG. 22, the color of the digital template 350 changes from red to green. In other embodiments, other aspects of the digital template 350 may change to provide the visual indication. In some embodiments, the AR device 30 may also provide or an audible indication via the speaker 58.

While the disclosure has been illustrated and described in detail in the drawings and foregoing description, such an illustration and description is to be considered as exemplary and not restrictive in character, it being understood that only illustrative embodiments have been shown and described and that all changes and modifications that come within the spirit of the disclosure are desired to be protected.

There are a plurality of advantages of the present disclosure arising from the various features of the method, apparatus, and system described herein. It will be noted that alternative embodiments of the method, apparatus, and system of the present disclosure may not include all of the features described yet still benefit from at least some of the advantages of such features. Those of ordinary skill in the art may readily devise their own implementations of the method, apparatus, and system that incorporate one or more of the features of the present invention and fall within the spirit and scope of the present disclosure as defined by the appended claims.

1. A method for a patient-specific surgical procedure, comprising:
preparing a request for a customized surgical plan,
receiving the customized surgical plan from a vendor,
loading the customized surgical plan onto an augmented reality device, and
performing an orthopaedic surgical procedure with the augmented reality device in accordance with the customized surgical plan, wherein performing the orthopaedic surgical procedure includes selecting a surgical instrument based on information displayed by the augmented reality device and operating the surgical instrument based on visual indications provided by the augmented reality device.

2. The method of claim 1, wherein the visual indications provided by the augmented reality device include a digital template of the patient's bone overlaying the patient's actual bone.

3. The method of claim 2, wherein the augmented reality device is operable to change the digital template when the surgical instrument is properly positioned relative to the patient's bone.

4. The method of claim 3, wherein the augmented reality device is operable to change a color of the digital template when the surgical instrument is properly positioned relative to the patient's bone.

5. The method of claim 1, wherein performing the orthopaedic surgical procedure includes positioning the surgical instrument to permit the augmented reality device to identify the surgical instrument.

6. The method of claim 1, wherein performing the orthopaedic surgical procedure with the augmented reality device includes accessing the customized surgical plan on the augmented reality device to display a three dimensional rendered image of a patient's bone.

7. The method of claim 6, wherein accessing the customized surgical plan on the augmented reality device to display the three dimensional rendered image of the patient's bone includes displaying a digital template of the patient's bone overlaying the patient's actual bone on a display of the augmented reality device.

8. The method of claim 7, further comprising operating the augmented reality device to change the display of the augmented reality device when the surgical instrument is properly positioned relative to the patient's bone.

9. The method of claim 8, wherein operating the augmented reality device to change the display includes changing a color of the digital template when the surgical instrument is properly positioned relative to the patient's bone.

10. A method of utilizing a patient-specific surgical plan, the method comprising:
operating a display to provide surgical plan information,
identifying a surgical instrument,
updating the surgical plan information based on the surgical instrument,
monitoring the position of the surgical instrument, and
providing an indication to the user when the surgical instrument is properly positioned relative to the patient's bone.

11. The method of claim 10, wherein the indication is a visual indication.

12. The method of claim 11, wherein the visual indication includes a digital template of a patient's bone.

13. The method of claim 12, wherein providing the visual indication includes changing an outline of the digital template to indicate to the user when the surgical instrument is properly positioned relative to the patient's bone.

14. The method of claim 10, wherein the surgical plan information includes patient-specific medical images.

15. The method of claim 10, wherein the patient's bone is a tibia.

16. The method of claim 10, wherein the patient's bone is a femur.

17. The method of claim 10, wherein the patient's bone is a pelvic bone.

18. A system for utilizing a patient-specific surgical plan, comprising:
a surgical instrument including a data tag, and
an augmented reality device including a display, a sensor operable to scan the data tag and generate an electronic signal, and a controller operable to:
(i) receive the electronic signal,
(ii) determine the position of the surgical instrument based on the electronic signal, and
(iii) operate the display to provide an indication to the user when the surgical instrument is properly positioned relative to a patient's bone.

19. The system of claim 18, wherein the controller is operable to operate the display to overlie a digital template of the patient's bone on a patient's actual bone.

20. The system of claim 19, wherein the controller is operable to operate the display to change the digital template when the surgical instrument is properly positioned relative to the patient's bone.
